(19) United States
(12) Patent Application Publication
(10) Pub. No.: US 2026/0306858 A1
(43) Pub. Date: Oct. 8, 2026

(54) DEFIBRILLATOR, DEFIBRILLATOR MANAGEMENT SYSTEM, AND NON-TRANSITORY COMPUTER READABLE STORAGE MEDIUM

(71) Applicant: NIHON KOHDEN CORPORATION, Tokyo (JP)

(72) Inventor: Yoko NATSUHARA, Saitama (JP)

(73) Assignee: NIHON KOHDEN CORPORATION, Tokyo (JP)

(21) Appl. No.: 19/462,197

(22) Filed: Jan. 28, 2026

(30) Foreign Application Priority Data
Feb. 17, 2025 (JP) 2025-023340

(51) Int. Cl. A61N 1/39; A61N 1/37

(52) U.S. Cl. CPC A61N 1/3904; A61N 1/3702; A61N 1/3925; A61N 1/3993

(57) ABSTRACT

A defibrillator for performing electrocardiogram analysis and discharge processing on a subject. The defibrillator includes a receiver configured to receive an abnormality notification indicating that an abnormality in heartbeat of the subject is detected, and a power supply controller configured to activate the defibrillator, in a case where the receiver receives the abnormality notification.

### CROSS-REFERENCE TO RELATED APPLICATIONS

This application is based on and claims priority under 35 USC 119 from Japanese Patent Application No. 2025-023340 filed on Feb. 17, 2025, the entire content of which is incorporated herein by reference.

### TECHNICAL FIELD

The presently disclosed subject matter relates to a defibrillator, a defibrillator management system, and a program.

Japan Resuscitation Council, "JRC Resuscitation Guidelines 2020," published by Igaku Shoin on Jun. 15, 2021 defines that chest compression and respirator for a subject are continued during a cardiopulmonary resuscitation (CPR) period after electrocardiogram analysis and discharge processing by an automated external defibrillator (AED).

Incidentally, when a subject has an abnormality in heartbeat, there is a need for a technique that can more quickly rescue the subject.

### SUMMARY OF INVENTION

Aspect of non-limiting embodiments of the present disclosure relates to provide a defibrillator, a defibrillator management system, and a non-transitory computer readable storage medium storing a program that can more quickly rescue a subject when the subject has an abnormality in heartbeat.

Aspects of certain non-limiting embodiments of the present disclosure address the features discussed above and/or other features not described above. However, aspects of the non-limiting embodiments are not required to address the above features, and aspects of the non-limiting embodiments of the present disclosure may not address features described above.

According to an aspect of the present disclosure, there is provided a defibrillator for performing electrocardiogram analysis and discharge processing on a subject, the defibrillator including:

a receiver configured to receive an abnormality notification indicating that an abnormality in heartbeat of the subject is detected; and

a power supply controller configured to activate the defibrillator, in a case where the receiver receives the abnormality notification.

### BRIEF DESCRIPTION OF DRAWINGS

Exemplary embodiment(s) of the present invention will be described in detail based on the following figures, wherein:

FIG. 1 illustrates an example of a defibrillator management system according to an embodiment of the presently disclosed subject matter;

FIG. 2 is a sequence diagram (part 1) illustrating the procedure of an operation of the defibrillator management system when an operation mode of a subject terminal illustrated in FIG. 1 is a home mode;

FIG. 3 is a sequence diagram (part 2) illustrating the procedure of the operation of the defibrillator management system when an operation mode of the subject terminal illustrated in FIG. 1 is the home mode;

FIG. 4 illustrates an example of an emergency screen displayed on a monitor or the like of the subject terminal illustrated in FIG. 1;

FIG. 5 illustrates an example of an emergency screen displayed on a monitor or the like of a rescuer terminal illustrated in FIG. 1;

FIG. 6 is a sequence diagram (part 1) illustrating the procedure of an operation of the defibrillator management system when an operation mode of the subject terminal illustrated in FIG. 1 is an outing mode;

FIG. 7 is a sequence diagram (part 2) illustrating the procedure of the operation of the defibrillator management system when an operation mode of the subject terminal illustrated in FIG. 1 is the outing mode; and

FIG. 8 illustrates an example of an emergency screen displayed on a monitor or the like of the rescuer terminal illustrated in FIG. 1.

### DESCRIPTION OF EMBODIMENTS

Hereinafter, a defibrillator management system according to the presently disclosed subject matter will be described with reference to the drawings. Members having the same reference numerals as those already described in the description of the embodiment will not be described for convenience of description. Further, for convenience of description, dimensions of each member illustrated in the drawings may be different from actual dimensions of each member.

### Overall Configuration of Defibrillator Management System

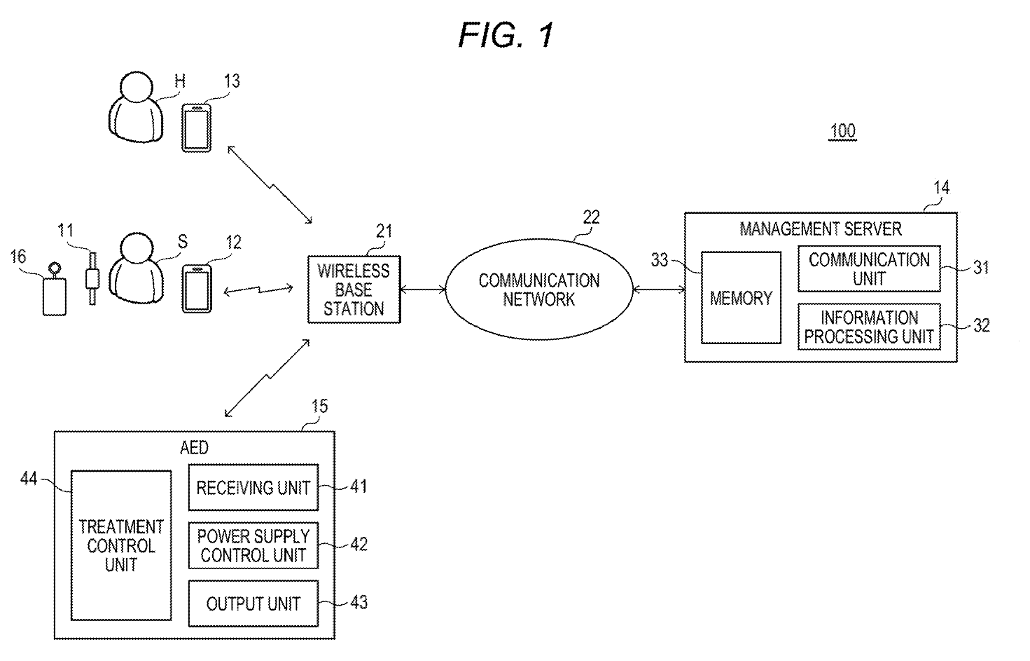

Here, a configuration of a defibrillator management system 100 according to the presently disclosed subject matter will be described. FIG. 1 illustrates an example of the defibrillator management system 100 according to an embodiment of the presently disclosed subject matter. As illustrated in FIG. 1, the defibrillator management system 100 includes a detection device 11, a subject terminal 12, a rescuer terminal 13, a management server 14, an AED (defibrillator) 15, and a notification device 16. The subject terminal 12, the rescuer terminal 13, and the management server 14 are examples of an information processing device.

The management server 14 can include a communication unit 31, an information processing unit 32, and a memory 33. The communication unit 31 is configured to communicate with each of the subject terminal 12, the rescuer terminal 13, and the AED 15, via a wireless base station 21 and a communication network 22.

The detection device 11 is, for example, a wearable device such as a smart watch, and is worn by a subject S who is suspected of having or is concerned about heart disease. More specifically, the detection device 11 is configured to measure any physiological information that can be measured from the upper arm or wrist of the subject S. More specifically, the detection device 11 is configured to measure physiological information such as heartbeat (for example, a high heart rate, a low heart rate, or an irregular heart rate), the presence or absence of arrhythmia from an electrocardiogram waveform, a pulse rate, and SpO2. Further, the detection device 11 is configured to acquire, for example, acceleration information of the subject S, and detect a fall of the subject S based on the acquired acceleration information.

Further, the detection device 11 is configured to detect an abnormality in the heartbeat of the subject S, that is, a situation in which discharge processing by the AED 15 is necessary, based on the measurement result of the physiological information of the subject S and the acceleration information. For example, in a case where the measurement result of the physiological information of the subject S exceeds a threshold value, in a case where a rate of change in the measurement result is equal to or greater than a predetermined value, or in a case where the subject S does not get up for a predetermined time or more after falling, the detection device 11 determines that the subject S has an abnormality in the heartbeat.

The subject terminal 12 is, for example, a portable terminal device used by the subject S, such as a smartphone, a phablet, or a tablet. The rescuer terminal 13 is a terminal device used by a rescuer H, such as a family member of the subject S, who is expected to rescue the subject S if the subject S experiences an abnormality. The rescuer terminal 13 is, for example, a portable terminal device such as a smartphone, a phablet, or a tablet, similar to the subject terminal 12.

The AED 15 is a device configured to perform electrocardiogram analysis and discharge processing, and is provided at the home of the subject S. The AED 15 is portable and can be placed in a bag or the like of the subject S when the subject S goes out.

More specifically, the AED 15 can include a receiving unit 41, a power supply control unit 42, an output unit 43, and a treatment control unit 44. The receiving unit 41 is configured to communicate with an external device such as the management server 14, via the wireless base station 21 and the communication network 22. The receiving unit 41 may be a communication interface. The power supply control unit 42 is configured to control the activation of the AED 15. The output unit 43 is configured to output guidance on how to use the AED 15 and the like. The output unit 43 may be an output interface. The treatment control unit 44 is configured to perform electrocardiogram analysis and discharge processing on the subject. The AED 15 may include a central processing unit (CPU), a read only memory (ROM), a random access memory (RAM), a hard disk drive (HDD), and the like. The CPU may function as the receiving unit 41, the power supply control unit 42, the output unit 43, and the treatment control unit 44 and the like.

The notification device 16 is owned by the subject S. The notification device 16 can include a light emitting unit and an output unit, and is a device configured to light up the light emitting unit and output a voice or a notification sound from the output unit. For example, the notification device 16 is in the form of a key chain and can be attached to a bag or the like of the subject S. Further, the notification device 16 is associated with the subject terminal 12. For example, the subject terminal 12 and the notification device 16 are paired with each other, and IDs (Identification) and the like are registered.

### Operation of Defibrillator Management System Pre-set

An application dedicated to the service provided by the defibrillator management system 100 is installed in the subject terminal 12 and the rescuer terminal 13, for example, by a preliminary operation by the subject S or the rescuer H.

The detection device 11, the subject terminal 12, and the rescuer terminal 13 are associated with each other in advance, and the association is registered in the memory 33 of the management server 14. For example, the memory 33 is configured to store a correspondence table indicating a set of IP addresses of the detection device 11, the subject terminal 12, and the rescuer terminal 13. The AED 15 is associated with each of the subject terminal 12 and the rescuer terminal 13, and the association is registered in the memory 33 of the management server 14.

### Mode Setting

For example, the subject terminal 12 can operate in a home mode that is an operation mode suitable for a case where the subject S is at home, and an outing mode that is an operation mode suitable for a case where the subject S is out. For example, the subject S sets the subject terminal 12 to operate in the home mode. In a case where the subject S is out, the subject S sets the subject terminal 12 to operate in the outing mode. In a case where the operation mode is switched, the subject terminal 12 displays the operation mode after the switching on a monitor or the like of the subject terminal 12, for example.

The operation mode of the subject terminal 12 may be switched automatically. For example, the subject terminal 12 may be configured to switch the operation mode from the home mode to the outing mode, in a case where a distance of the subject terminal 12 from a designated location (for example, the home of the subject S) is equal to or greater than a predetermined value.

Further, the AED 15 may be configured to detect whether the subject S is out. For example, the AED 15 is equipped with a global positioning system (GPS) receiver, and is configured to detect the position of the AED 15 based on a GPS signal received by the GPS receiver. Then, it is assumed that the distance of the AED 15 from the designated location (for example, the home of the subject S) is equal to or greater than the predetermined value. In this case, the AED 15 transmits to the subject terminal 12 an out-of-home detection signal indicating that the subject S is out, so as to notify the subject terminal 12 that the operation mode is to be switched from the home mode to the outing mode. Upon receiving the out-of-home detection signal from the AED 15, the subject terminal 12 switches the operation mode from the home mode to the outing mode.

Further, a communication device may be provided in a storage case of the AED 15 installed in the home of the subject S, and communication may be performed between the communication device and the AED 15 according to the Bluetooth (registered trademark) standard. In this case, for example, the AED 15 may transmit the out-of-home detection signal to the subject terminal 12 when communication with the communication device is disconnected.

The AED 15 may also be configured to detect that the AED 15 has returned to the home of the subject S. For example, in a case where the operation mode of the subject terminal 12 is the outing mode, it is assumed that the distance between the position of the

AED 15 based on the GPS signal and the designated location changes to less than a predetermined value. In this case, the AED 15 transmits to the subject terminal 12 a return detection signal indicating that the subject S is back home, so as to notify the subject terminal 12 that the operation mode is to be switched from the outing mode to the home mode. Further, for example, in a case where the operation mode of the subject terminal 12 is the outing mode, the AED 15 may transmit the return detection signal to the subject terminal 12 when communication with the communication device provided in the storage case of the AED 15 installed at home is started.

### Example of Operation Procedure for Rescue Process in a case where Home Mode is Set

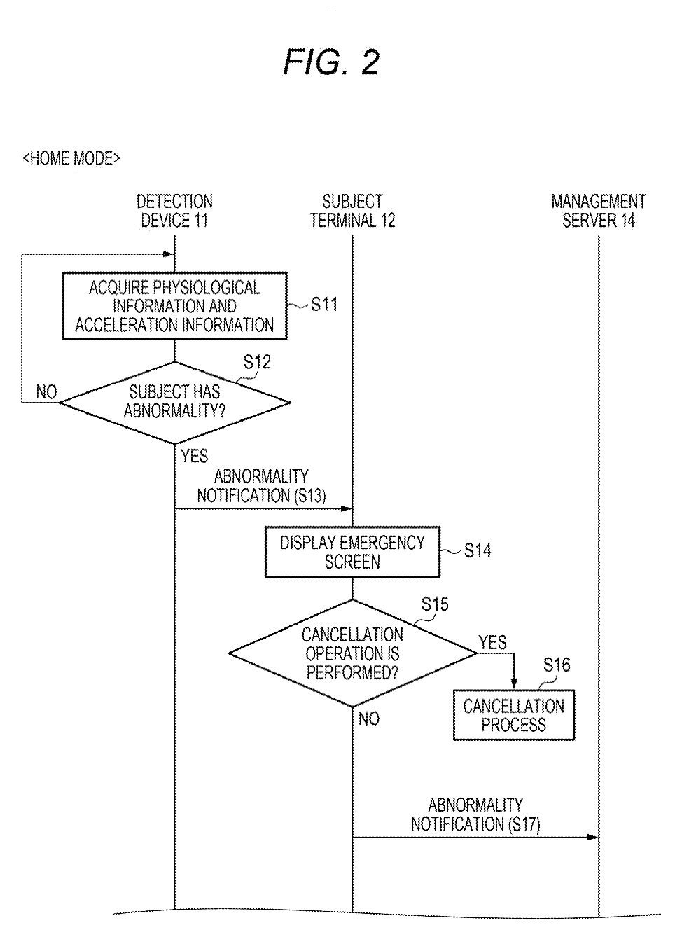

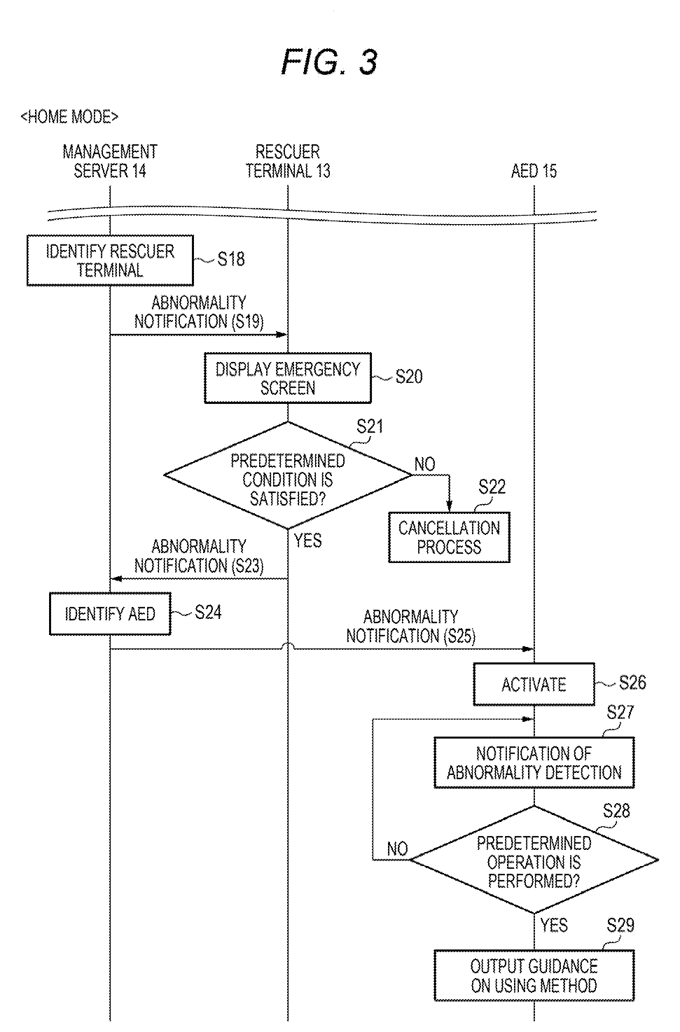

FIGS. 2 and 3 are sequence diagrams illustrating the procedure of the operation of the defibrillator management system 100 in a case where the operation mode of the subject terminal 12 illustrated in FIG. 1 is the home mode.

### (a) Operation of Detection Device and Subject Terminal

First, referring to FIGS. 1 and 2, the detection device 11 acquires, for example, physiological information and acceleration information of the subject S (step S11). Then, the detection device 11 determines whether the subject S has an abnormality in the heartbeat, based on the acquired physiological information and acceleration information (step S12). If the detection device 11 determines that the subject S does not have abnormality in the heartbeat ("NO" in step S12), the detection device 11 continues to acquire the physiological information and the acceleration information (step S11).

On the other hand, if the detection device 11 determines that the subject S is having an abnormality in the heartbeat ("YES" in step S12), the detection device 11 transmits, to the subject terminal 12 associated with the detection device 11, an abnormality notification indicating that an abnormality in the heartbeat of the subject S has been detected (step S13).

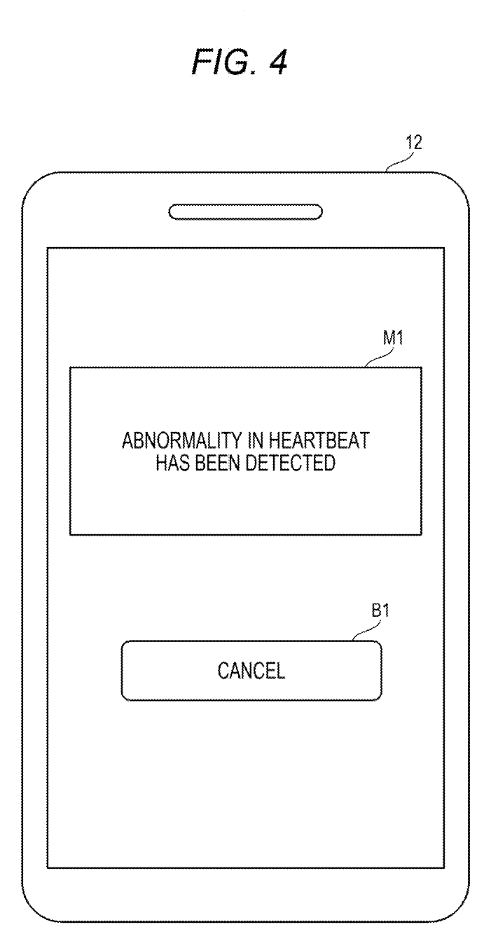

Next, in a case where the subject terminal 12 receives the abnormality notification transmitted from the detection device 11, the subject terminal 12 outputs a notification sound and displays an emergency screen on a monitor or the like of the subject terminal 12, for example (step S14). FIG. 4 illustrates an example of the emergency screen displayed on the monitor or the like of the subject terminal 12 illustrated in FIG. 1. For example, as illustrated in FIG. 4, the emergency screen may include a message M1 indicating that an abnormality in the heartbeat of the subject S has been detected, and a cancel button B1.

If the subject S determines that there is nothing abnormal with itself, the subject S performs an operation to select the cancel button B1 included in the emergency screen

(hereinafter referred to as a "cancellation operation"). As described above, when the cancellation operation is performed on the subject terminal 12 ("YES" in step S15 illustrated in FIG. 2), the subject terminal 12 performs a cancellation process such as terminating the display of the emergency screen (step S16).

In a case where the detection device 11 has a monitor or the like, the emergency screen may be displayed on the monitor or the like of the detection device 11. In this case, the subject S can also perform the cancellation operation on the detection device 11.

On the other hand, it is assumed that no cancellation operation is performed ("NO" in step S15) during a time period from when the subject terminal 12 receives the abnormality notification (step S13) until a predetermined time has elapsed. In this case, the subject terminal 12 transmits an abnormality notification to the management server 14 (step S17).

### (b) Operations of Management Server and Rescuer Terminal

Next, with reference to FIGS. 1 and 3, in a case where the abnormality notification transmitted from the subject terminal 12 is received by the communication unit 31, the information processing unit 32 of the management server 14 identifies the rescuer terminal 13 associated with the subject terminal 12 that is the transmission source of the abnormality notification (step S18). Then, the information processing unit 32 transmits an abnormality notification to the identified rescuer terminal 13, via the communication unit 31 (step S19).

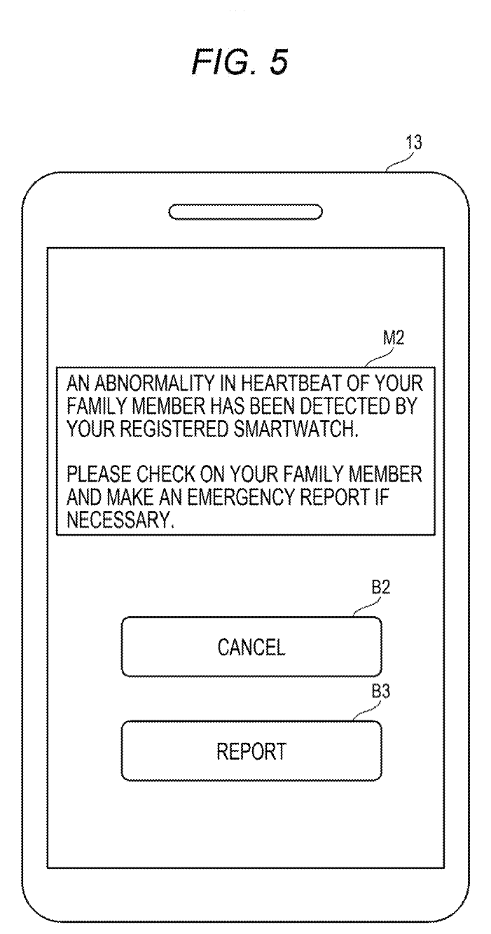

Next, in a case where the rescuer terminal 13 receives the abnormality notification transmitted from the management server 14, the rescuer terminal 13 outputs a notification sound and displays an emergency screen on the monitor or the like of the rescuer terminal 13, for example (step S20). FIG. 5 illustrates an example of the emergency screen displayed on the monitor or the like of the rescuer terminal 13 illustrated in FIG. 1. For example, as illustrated in FIG. 5, the emergency screen may include a message M2 indicating that an abnormality in the heartbeat of the subject S has been detected, a cancel button B2, and a report button B3.

In a case where the rescuer H sees the emergency screen displayed on the monitor or the like of the rescuer terminal 13, the rescuer checks the condition of the subject S, for example, and determines whether the subject S has an abnormality. In a case where the rescuer H determines that there is nothing abnormal with the subject S, the rescuer performs a cancellation operation on the rescuer terminal 13. As described above, in a case where the cancellation operation is performed on the rescuer terminal 13, the rescuer terminal 13 determines that a predetermined condition is not satisfied ("NO" in step S21). The rescuer terminal 13 performs a cancellation process, such as terminating the display of the emergency screen (step S22).

In a case where the rescuer H carries a wearable device such as a smart watch associated with the rescuer terminal 13, the rescuer terminal 13 may transmit an abnormality notification to the wearable device. In such a case, the rescuer H can also perform the cancellation operation on the emergency screen displayed on the wearable device.

On the other hand, in a case where the rescuer H determines that the subject S has an abnormality, the rescuer H performs an operation to select the report button B3 on the rescuer terminal 13 (hereinafter referred to as a "report operation"). In this case, the rescuer terminal 13 starts to call an emergency call destination that can call an ambulance, for example. This allows the rescuer H to easily report to the emergency call destination. If the emergency screen is displayed on the wearable device of the rescuer, the rescuer H can also perform the report operation on the emergency screen displayed on the wearable device.

As described above, in a case where the report operation is performed on the rescuer terminal 13 or the wearable device of the rescuer H, the rescuer terminal 13 determines that the predetermined condition is satisfied ("YES" in step S21). Then, the rescuer terminal 13 transmits an abnormality notification to the management server 14 (step S23).

Further, it is assumed that neither the cancellation operation nor the report operation is performed during a time period from when the rescuer terminal 13 receives the abnormality notification (step S19) until a predetermined time has elapsed. In this case as well, the rescuer terminal 13 determines that the predetermined condition is satisfied ("YES" in step S21) and transmits an abnormality notification to the management server 14 (step S23).

Next, in a case where the abnormality notification transmitted from the rescuer terminal 13 is received by the communication unit 31, the information processing unit 32 of the management server 14 identifies the AED 15 associated with the rescuer terminal 13 (step S24). Then, the information processing unit 32 transmits an abnormality notification to the identified AED 15, via the communication unit 31 (step S25).

The rescuer terminal 13 may be configured to transmit the abnormality notification to the AED 15, without using the management server 14. In other words, in a case where the rescuer terminal 13 determines that the predetermined condition is satisfied ("YES" in step S21), the rescuer terminal 13 may transmit the abnormality notification to the AED 15 associated with the rescuer terminal 13, instead of transmitting the abnormality notification to the management server 14.

### (c) Operation of AED

The AED 15 is on standby in, for example, a sleep mode. In a case where the receiving unit 41 receives the abnormality notification from the management server 14 or the rescuer terminal 13, the power supply control unit 42 of the AED 15 cancels the sleep mode and activates the AED 15 (step S26). As described above, in a case where the subject S has an abnormality in the heartbeat, the AED 15 can be activated before the rescuer H touches the AED 15. Therefore, the rescue of the subject S can be performed more quickly.

The output unit 43 of the AED 15 issues a notification indicating that the abnormality of the subject S is detected (step S27). For example, the output unit 43 is configured to output, as a notification indicating that an abnormality has been detected, a voice message or a notification sound such as "An abnormality has been detected by an app linked to the AED, and the AED has been turned on. Please check the patient's condition. If the patient is not conscious or breathing, please call an emergency service and use this AED." In such a case, the rescuer terminal 13 may also output a voice message such as "The registered AED has been activated."

Here, the time period when abnormalities related to heartbeats such as arrhythmia occur is often from midnight to dawn. Therefore, even if the subject S has an abnormality in the heartbeat, the rescuer H may not be able to notice the abnormality. In addition, even in a case where the rescuer H can notice the abnormality, there is a possibility that the rescuer H may become agitated and lose track of the location of the AED 15, and may not be able to quickly begin life-saving treatment using the AED 15.

In this regard, in the configuration as described above, since the voice message or the notification sound is output from the AED 15, the rescuer H can not only notice the abnormality of the subject S but also easily grasp the location of the AED 15, and can start using the AED 15 more quickly.

Next, it is assumed that after the notification of the abnormality detection by the AED 15 (step S27), a predetermined operation by touching the AED 15 such as opening the lid is not performed ("NO" in step S28). In this case, the output unit 43 of the AED 15 repeats the notification of the abnormality detection (step S27).

On the other hand, it is assumed that the predetermined operation is performed by the rescuer H ("YES" in step S28). In this case, the output unit 43 of the AED 15 outputs guidance on how to use the AED 15, for example, how to attach pads (step S29).

That is, until the rescuer H directly touches the AED 15, the notification is performed to inform people around that an abnormality in the subject S has been detected and the position of the AED 15. When the rescuer H can directly operate the AED 15, the notification from the AED 15 is switched to the guidance on how to use the AED 15. Therefore, the rescuer H can be encouraged to use the AED 15 more effectively.

### Example of Operation Procedure for Rescue Process in a Case Where Outing Mode is Set

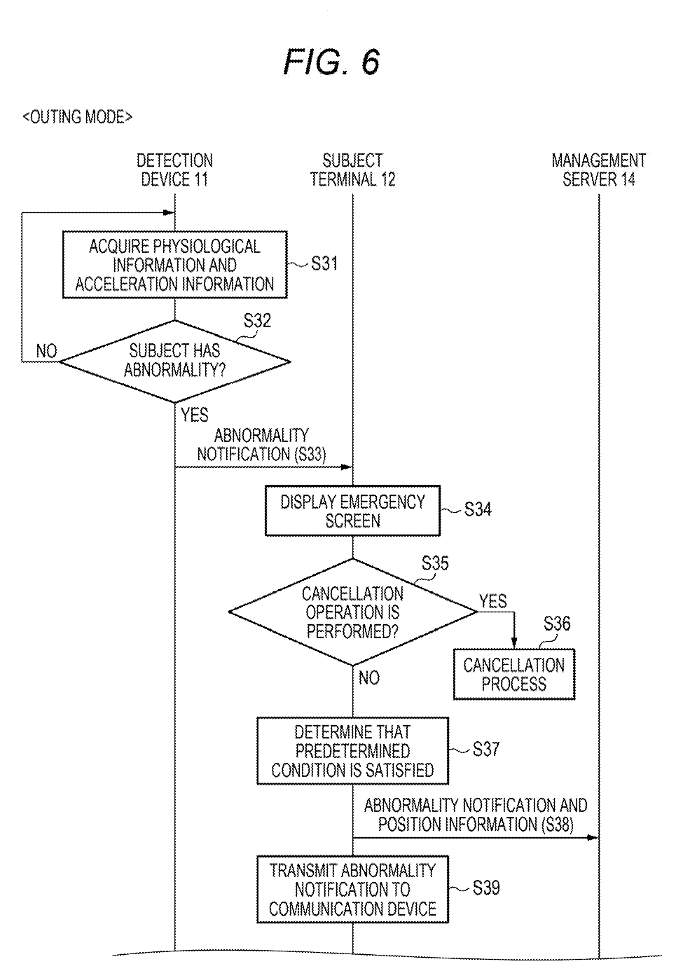

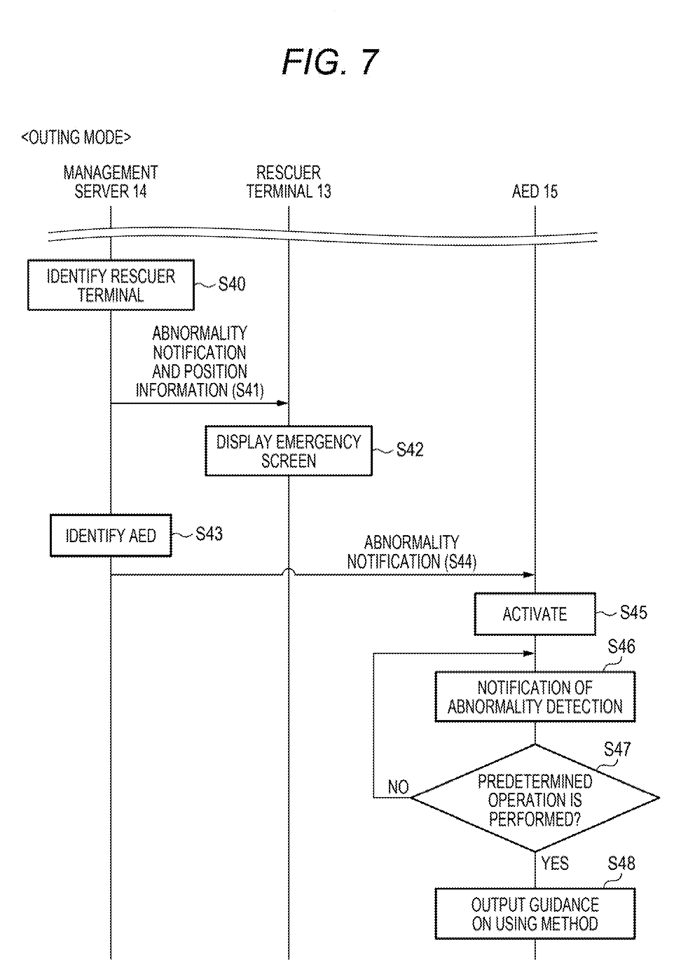

FIGS. 6 and 7 are sequence diagrams illustrating the procedure of the operation of the defibrillator management system 100 when the operation mode of the subject terminal 12 illustrated in FIG. 1 is the outing mode. The following description will be given on the assumption that the subject S goes out while carrying the AED 15, and the subject S and the rescuer H are in different locations.

### (a) Operation of Subject Terminal

Since operations from step S31 to step S34 illustrated in FIG. 6 are the same as or similar to the operations from step S11 to step S14 illustrated in FIG. 2, a detailed description thereof will not be repeated here.

Referring to FIGS. 1 and 6, it is assumed that a cancellation operation is performed on the subject terminal 12 in a state where the emergency screen is displayed on a monitor or the like of the subject terminal 12 ("YES" in step S35). In this case, the subject terminal 12 performs a cancellation process, such as terminating the display of the emergency screen (step S36).

On the other hand, it is assumed that no cancellation operation is performed ("NO" in step S35) during a time period from when the subject terminal 12 receives the abnormality notification (step S33) until a predetermined time has elapsed. In this case, the subject terminal 12 determines that a predetermined condition is satisfied (step S37). Then, the subject terminal 12 transmits the abnormality notification and position information indicating the position of the subject terminal 12 to the management server 14 (step S38).

The subject terminal 12 transmits an abnormality notification to the notification device 16 associated with the subject terminal 12 (step S39). In addition, the abnormality notification may be transmitted to the notification device 16 directly by the detection device 11 without using the subject terminal 12.

When receiving the abnormality notification from the subject terminal 12 or the detection device 11, the notification device 16 notifies that the abnormality of the subject S has been detected, for example, by turning on or blinking a light emitting unit provided in the notification device 16. Further, the notification device 16 outputs a voice message such as "Please take out the AED from your bag and use it." This makes people around the subject S aware of the abnormality of the subject S and encourages them to use the AED 15.

Instead of the notification device 16 or in addition to the notification device 16, the subject terminal 12 may output a voice message such as "Please take out the AED from your bag and use it."

### (b) Operations of Management Server and Rescuer Terminal

Next, with reference to FIGS. 1 and 7, in a case where the abnormality notification and the position information transmitted from the subject terminal 12 are received by the communication unit 31, the information processing unit 32 of the management server 14 identifies the rescuer terminal 13 associated with the subject terminal 12 that is the transmission source of the abnormality notification (step S40). Then, the information processing unit 32 transmits the abnormality notification and the position information to the identified rescuer terminal 13 via the communication unit 31 (step S41).

Next, in a case where the rescuer terminal 13 receives the abnormality notification and the position information transmitted from the management server 14, the rescuer terminal 13 outputs a notification sound and displays an emergency screen on the monitor or the like of the rescuer terminal 13, for example (step S42).

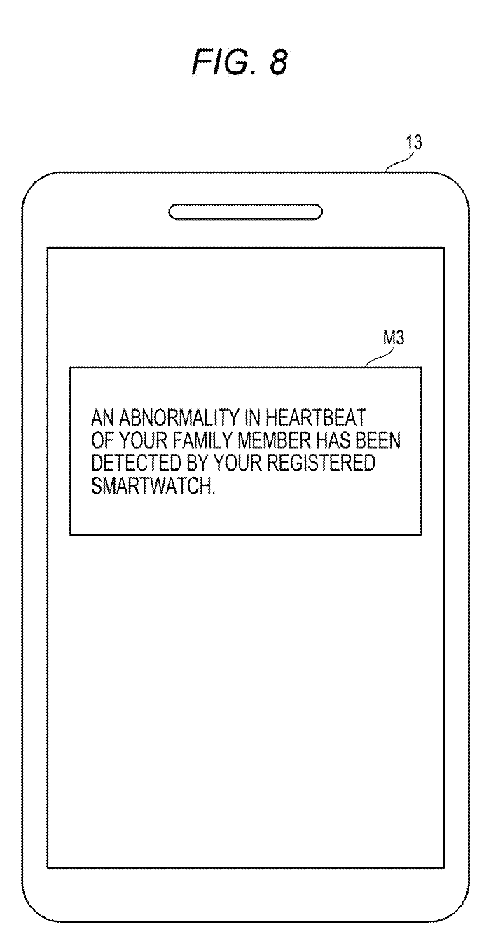

FIG. 8 illustrates an example of the emergency screen displayed on the monitor or the like of the rescuer terminal 13 illustrated in FIG. 1. For example, the emergency screen may include a message M3 indicating that an abnormality in the heartbeat of the subject S has been detected. By looking at the emergency screen displayed on the rescuer terminal 13, the rescuer H can understand that the subject S has an abnormality and the position of the subject S.

Referring to FIGS. 1 and 7, operation from step S43 to step S48 of FIG. 7 is the same as or similar to the operation from step S24 to step S29 of FIG. 3, and thus detailed description thereof will not be repeated here.

Here, as illustrated in FIGS. 2 and 3, in a case where the subject terminal 12 is set to the home mode, if the cancellation operation is not performed on the subject terminal 12 ("NO" in step S15) and the report operation is performed on the rescuer terminal 13, or in a case where neither the cancellation operation nor the report operation is performed during the time period from when the rescuer terminal 13 receives the abnormality notification until a predetermined time has elapsed, the rescuer terminal 13 determines that the predetermined condition is satisfied ("YES" in step S21).

That is, in a case where the subject S is at home, the activation of the AED 15 is controlled according to whether the rescuer H such as a family member operates the rescuer terminal 13. Therefore, for example, the AED 15 can be activated after the necessity of activating the AED 15 is more appropriately determined.

In this regard, as illustrated in FIG. 6, in a case where the subject terminal 12 is set to the outing mode, if the cancellation operation is not performed on the subject terminal 12 ("NO" in step S35), it is determined that the predetermined condition is satisfied by the subject terminal 12, regardless of whether a specified operation is performed on the rescuer terminal 13 (step S37).

That is, in a case where the subject S is out, the activation of the AED 15 is controlled depending on whether the subject S operates the subject terminal 12. Therefore, even if there is no rescuer H such as a family member near the subject S, the AED 15 can be activated.

As described above, in the defibrillator management system 100, since the activation condition of the AED 15 and the method of notification to those around the subject S can be changed between the case where the subject S is at home and the case where the subject S is out, it is possible to perform a more appropriate operation according to the situation of the subject S.

Although the embodiments of the presently disclosed subject matter have been described above, the technical scope of the presently disclosed subject matter should not be construed as being limited to the description of the present embodiments. The present embodiments are merely examples, and it is understood by those skilled in the art that various modifications of the embodiments are possible within the scope of the disclosed subject matter described in the claims. The technical scope of the presently disclosed subject matter should be determined based on the scope of the disclosed subject matter described in the claims and equivalents thereof.

The processes of the information processing device of the defibrillator management system 100 according to the embodiment can be implemented as a computer program that operates in the information processing device. That is, each of the subject terminal 12, the rescuer terminal 13, and the management server 14 may include a processor such as a CPU.

new paragraph...

The program is stored in a non-transitory computer-readable medium and can be read by a computer. Examples of the non-transitory computer-readable medium include a magnetic recording medium, a magneto-optical recording medium, a CD-ROM, a CD-R, a CD-R/W, and a semi-conductor memory (including an EPROM and a flash ROM). The program may be read by a computer using various types of temporary computer-readable media. Examples of the temporary computer-readable medium include an electric signal, an optical signal, and an electromagnetic wave. The temporary computer-readable medium can supply a program to the computer via a wired communication path such as an electric wire and an optical fiber or a wireless communication path.

As described above, the present specification discloses the following matters. (1) A defibrillator that performs electrocardiogram analysis and discharge processing on a subject, the defibrillator including:

a receiving unit configured to receive an abnormality notification indicating that an abnormality in heartbeat of the subject is detected; and

a power supply control unit configured to activate the defibrillator when the receiving unit receives the abnormality notification.

With such a configuration, if the subject has an abnormality in the heartbeat, the defibrillator can be activated before the rescuer touches the defibrillator. Therefore, the rescue of the subject can be performed more quickly.

(2) The defibrillator according to (1), further including:

an output unit configured to notify that an abnormality of the subject is detected when the defibrillator is activated as a result of the receiving unit receiving the abnormality notification.

With such a configuration, people around the defibrillator can be notified that the subject is having an abnormality, and can also be informed of the position of the defibrillator.

(3) In the defibrillator according to (2), the output unit outputs guidance on how to use the defibrillator when a predetermined operation is performed by a rescuer touching the defibrillator.

In this way, until the rescuer directly touches the defibrillator, the notification is performed to inform people around that an abnormality in the subject has been detected and the position of the defibrillator, and when the rescuer is in a state of being able to directly operate the defibrillator, the notification from the defibrillator is switched to the guidance on how to use it, so that the rescuer can be more appropriately supported.

(4) In the defibrillator according to any one of (1) to (3),

a subject terminal, which is a terminal device of the subject, is configured to receive settings of a home mode that is an operation mode suitable for a case where the subject is at home and an outing mode that is an operation mode suitable for a case where the subject is out,

in a case where the operation mode of the subject terminal is set to the home mode, transmission of the abnormality notification to the defibrillator is canceled depending on whether a predetermined operation is performed on a terminal device of a rescuer of the subject,

in a case where the operation mode of the subject terminal is set to the outing mode, transmission of the abnormality notification to the defibrillator is canceled depending on whether a predetermined operation is performed on the subject terminal, and

the defibrillator notifies the subject terminal of switching the operation mode from the home mode to the outing mode when a distance of the defibrillator from a designated location is equal to or greater than a predetermined value.

With such a configuration, when the subject is at home, the abnormality notification is transmitted to the defibrillator in response to the operation on the terminal device by the rescuer such as a family member, and thus, for example, the defibrillator can be activated after the necessity of activating the defibrillator is more appropriately determined.

On the other hand, when the subject is out, the abnormality notification is transmitted to the defibrillator in response to the presence or absence of the operation on the terminal device by the subject, so that the defibrillator can be activated even if there is no rescuer such as a family member nearby.

In addition, even if the subject does not manually operate the subject terminal when the subject goes out, the operation mode can be automatically switched to the outing mode.

(5) In the defibrillator according to (4), in the case where the operation mode of the subject terminal is set to the outing mode, the defibrillator notifies the subject terminal of switching the operation mode from the outing mode to the home mode when the distance of the defibrillator from the designated location changes to less than a predetermined value.

With such a configuration, even if the subject does not manually operate the subject terminal when the subject returns home, the operation mode can be automatically switched to the home mode.

(6) In the defibrillator according to (4) or (5), the defibrillator determines whether the distance of the defibrillator from the designated location is equal to or greater than a predetermined value based on at least one of a position of the defibrillator detected based on a GPS signal provided in the defibrillator, and presence or absence of communication connection between a communication device provided at the designated location and the defibrillator.

With such a configuration, it is possible to detect when the subject goes out or returns home without performing complicated processing.

(7) A defibrillator management system including:

a defibrillator configured to perform electrocardiogram analysis and discharge processing on a subject;

a detection device configured to detect an abnormality in heartbeat of the subject and transmit an abnormality notification; and

an information processing device configured to receive the abnormality notification,

in which the information processing device transmits the abnormality notification to the defibrillator when a predetermined condition is satisfied after receiving the abnormality notification.

With such a configuration, if the subject has an abnormality in the heartbeat, the defibrillator can be activated before the rescuer touches the defibrillator. Therefore, the rescue of the subject can be performed more quickly.

(8) In the defibrillator management system according to (7), the detection device is a wearable device worn by the subject.

With such a configuration, for example, it is possible to acquire physiological information such as the heartbeat and SpO2 of the subject, acceleration information indicating a falling state of the subject, and the like, and by using the acquired information, an abnormality in the heartbeat of the subject can be detected more accurately.

(9) In the defibrillator management system according to (7) or (8),

the information processing device is a terminal device associated with the detection device, and

when a report is made in response to an operation by the subject or a rescuer after receiving the abnormality notification or when a cancellation operation is not performed by the subject or the rescuer within a predetermined time after receiving the abnormality notification, the terminal device determines that the predetermined condition is satisfied and transmits the abnormality notification to the defibrillator.

With such a configuration, for example, the defibrillator can be activated in response to the subject or rescuer using the terminal device to report to the emergency call destination that can call an ambulance. Further, for example, if the subject or rescuer determines that the use of the defibrillator is unnecessary, the subject or rescuer can prevent the defibrillator from being activated unnecessarily by performing the cancellation operation on the terminal device. Further, since the abnormality notification is transmitted from the terminal device to the defibrillator, the defibrillator can be activated more quickly.

(10) In the defibrillator management system according to (7) or (8),

the information processing device is a management server configured to communicate with a terminal device associated with the detection device,

the management server transmits the abnormality notification to the terminal device once receiving the abnormality notification,

when a report is made in response to an operation by the subject or a rescuer after receiving the abnormality notification or when a cancellation operation is not performed by the subject or the rescuer within a predetermined time after receiving the abnormality notification, the terminal device determines that the predetermined condition is satisfied and transmits the abnormality notification to the management server, and

the management server transmits the abnormality notification to the defibrillator once receiving the abnormality notification from the terminal device.

With such a configuration, for example, the defibrillator can be activated in response to the subject or rescuer using the terminal device to report to the emergency call destination that can call an ambulance. Further, for example, if the subject or rescuer determines that the use of the defibrillator is unnecessary, the subject or rescuer can prevent the defibrillator from being activated unnecessarily by performing the cancellation operation on the terminal device. Further, even in a configuration in which the abnormality notification cannot be directly transmitted from the terminal device to the defibrillator, the abnormality notification can be transmitted to the defibrillator via the management server.

(11) In the defibrillator management system according to any one of (7) to (10),

the detection device is associated with a subject terminal that is a terminal device of the subject, and

the subject terminal is configured to receive settings of a home mode that is an operation mode suitable for a case where the subject is at home and an outing mode that is an operation mode suitable for a case where the subject is out.

With such a configuration, for example, since the activation condition of the defibrillator and the method of notification to those around the subject can be changed between the case where the subject is at home and the case where the subject is out, it is possible to perform a more appropriate operation according to the situation of the subject.

(12) In the defibrillator management system according to (11),

the subject terminal is associated with a rescuer terminal that is a terminal device of a rescuer of the subject,

in a case where the operation mode of the subject terminal is set to the home mode, whether the predetermined condition is satisfied is determined depending on presence or absence of a predetermined operation on the rescuer terminal, and

in a case where the operation mode of the subject terminal is set to the outing mode, whether the predetermined condition is satisfied is determined depending on presence or absence of a predetermined operation on the subject terminal.

With such a configuration, when the subject is at home, the abnormality notification is transmitted to the defibrillator in response to the operation on the terminal device by the rescuer such as a family member, and thus, for example, the defibrillator can be activated after the necessity of activating the defibrillator is more appropriately determined.

On the other hand, when the subject is out, the abnormality notification is transmitted to the defibrillator in response to the presence or absence of the operation on the terminal device by the subject, so that the defibrillator can be activated even if there is no rescuer such as a family member nearby.

(13) In the defibrillator management system according to (11) or (12), when a distance of the subject terminal or the defibrillator from a designated location is equal to or greater than a predetermined value, the subject terminal switches the operation mode from the home mode to the outing mode.

With such a configuration, even if the subject does not manually operate the subject terminal, the operation mode can be automatically switched.

(14) The defibrillator management system according to any one of (7) to (13), further including:

a notification device to be used by the subject and associated with the detection device or a terminal device of the subject, in which

the detection device transmits the abnormality notification to the notification device directly or via the terminal device when detecting an abnormality of the subject, and

the notification device notifies that an abnormality of the subject is detected when receiving the abnormality notification.

With such a configuration, if the subject has an abnormality, the notification device outputs a sound or the like, making it possible to cause people around the subject to notice the abnormality of the subject.

(15) A program used in an information processing device included in a defibrillator management system,

the information processing device being communicative with a defibrillator that performs electrocardiogram analysis and discharge processing on a subject,

the program causing a computer to execute:

a step of receiving an abnormality notification indicating that an abnormality in heartbeat of the subject is detected; and

a step of transmitting the abnormality notification to the defibrillator when a predetermined condition is satisfied after the abnormality notification is received.

With such a configuration, if the subject has an abnormality in the heartbeat, the defibrillator can be activated before the rescuer touches the defibrillator. Therefore, the rescue of the subject can be performed more quickly.

What is claimed is:

1. A defibrillator for performing electrocardiogram analysis and discharge processing on a subject, the defibrillator comprising:
a receiver configured to receive an abnormality notification indicating that an abnormality in heartbeat of the subject is detected; and
a power supply controller configured to activate the defibrillator, in a case where the receiver receives the abnormality notification.

2. The defibrillator according to claim 1, further comprising:
an output unit configured to notify that an abnormality of the subject is detected, in a case where the defibrillator is activated as a result of the receiver receiving the abnormality notification.

3. The defibrillator according to claim 2,
wherein the output unit is configured to output guidance on how to use the defibrillator, in a case where a predetermined operation is performed by a rescuer touching the defibrillator.

4. The defibrillator according to claim 1,
wherein a subject terminalis configured to receive settings of a home mode that is an operation mode suitable for a case where the subject is at home and an outing mode that is an operation mode suitable for a case where the subject is out, the subject terminal being a terminal device of the subject,
wherein in a case where the operation mode of the subject terminal is set to the home mode, transmission of the abnormality notification to the defibrillator is canceled depending on whether a predetermined operation is performed on a terminal device of a rescuer of the subject,
wherein in a case where the operation mode of the subject terminal is set to the outing mode, transmission of the abnormality notification to the defibrillator is canceled depending on whether a predetermined operation is performed on the subject terminal, and
wherein the defibrillator is configured to notify the subject terminal of switching the operation mode from the home mode to the outing mode, in a case where a distance of the defibrillator from a designated location is equal to or greater than a predetermined value.

5. The defibrillator according to claim 4,
wherein in the case where the operation mode of the subject terminal is set to the outing mode, the defibrillator is configured to notify the subject terminal of switching the operation mode from the outing mode to the home mode, in a case where the distance of the defibrillator from the designated location changes to less than a predetermined value.

6. The defibrillator according to claim 4,
wherein the defibrillator is configured to determine whether the distance of the defibrillator from the designated location is equal to or greater than a predetermined value, based on at least one of a position of the defibrillator detected based on a GPS signal provided in the defibrillator, and presence or absence of communication connection between and the defibrillator and a communication device provided at the designated location.

7. A defibrillator management system comprising:
a defibrillator configured to perform electrocardiogram analysis and discharge processing on a subject;
a detection device configured to detect an abnormality in heartbeat of the subject and transmit an abnormality notification; and
an information processing device configured to receive the abnormality notification,
wherein the information processing device is configured to transmit the abnormality notification to the defibrillator, in a case where a predetermined condition is satisfied after receiving the abnormality notification.

8. The defibrillator management system according to claim 7,
wherein the detection device is a wearable device worn by the subject.

9. The defibrillator management system according to claim 7,
wherein the information processing device is a terminal device associated with the detection device, and
wherein in a case where a report is made in response to an operation by the subject or a rescuer after receiving the abnormality notification or in a case where a cancellation operation is not performed by the subject or the rescuer within a predetermined time after receiving the abnormality notification, the terminal device is configured to determine that the predetermined condition is satisfied and transmit the abnormality notification to the defibrillator.

10. The defibrillator management system according to claim 7,
wherein the information processing device is a management server configured to communicate with a terminal device associated with the detection device,
wherein the management server is configured to transmit the abnormality notification to the terminal device, in a case where the management server receives the abnormality notification,
wherein in a case where a report is made in response to an operation by the subject or a rescuer after receiving the abnormality notification or in a case where a cancellation operation is not performed by the subject or the rescuer within a predetermined time after receiving the abnormality notification, the terminal device is configured to determine that the predetermined condition is satisfied and transmit the abnormality notification to the management server, and
wherein the management server is configured to transmit the abnormality notification to the defibrillator, in a case where the management server receives the abnormality notification from the terminal device.

11. The defibrillator management system according to claim 7,
wherein the detection device is associated with a subject terminal that is a terminal device of the subject, and
wherein the subject terminal is configured to receive settings of a home mode that is an operation mode suitable for a case where the subject is at home and an outing mode that is an operation mode suitable for a case where the subject is out.

12. The defibrillator management system according to claim 11,
wherein the subject terminal is associated with a rescuer terminal that is a terminal device of a rescuer of the subject,
wherein in a case where the operation mode of the subject terminal is set to the home mode, whether the predetermined condition is satisfied is determined depending on presence or absence of a predetermined operation on the rescuer terminal, and
wherein in a case where the operation mode of the subject terminal is set to the outing mode, whether the predetermined condition is satisfied is determined depending on presence or absence of a predetermined operation on the subject terminal.

13. The defibrillator management system according to claim 11,
wherein in a case where a distance of the subject terminal or the defibrillator from a designated location is equal to or greater than a predetermined value, the subject terminal is configured to switch the operation mode from the home mode to the outing mode.

14. The defibrillator management system according to claim 7, further comprising:
a notification device to be used by the subject and associated with the detection device or a terminal device of the subject,
wherein the detection device is configured to transmit the abnormality notification to the notification device directly or via the terminal device, in a case where the detection device detects an abnormality of the subject, and
wherein the notification device is configured to notify that an abnormality of the subject is detected, in a case where the notification device receives the abnormality notification.

15. A non-transitory computer readable storage medium storing a program used in an information processing device included in a defibrillator management system,
the information processing device being configured to communicate with a defibrillator configured to perform electrocardiogram analysis and discharge processing on a subject,
the program is configured to cause a computer to execute:
receiving an abnormality notification indicating that an abnormality in heartbeat of the subject is detected; and
transmitting the abnormality notification to the defibrillator, in a case where a predetermined condition is satisfied after the abnormality notification is received.
